(19) United States
(12) Patent Application Publication
(10) Pub. No.: US 2026/0243758 A1
(43) Pub. Date: Aug. 20, 2026

(54) METHODS AND COMPOSITIONS FOR FUNCTIONALIZING THE SURFACE OF ELASTOMERIC PARTICLES

(71) Applicant: The Regents of the University of Colorado, a body corporate, Dener, CO (US)

(72) Inventors: Charles Shields, Longmont, CO (US); Cooper Thorne, Boulder, CO (US)

(21) Appl. No.: 19/162,389

(22) PCT Filed: Mar. 6, 2024
(86) PCT No.: PCT/US24/18685

§ 371 Date: Sep. 4, 2025

Related U.S. Application Data
(60) Provisional application No. 63/450,184, filed on Mar. 6, 2023.

(51) Int. Cl. G01N 33/543; G01N 33/82

(52) U.S. Cl. CPC G01N 33/54313; G01N 33/82

(57) ABSTRACT

The invention includes systems and methods to chemically modify, and specifically functionalize the surfaces of elastomeric particles that can be used in various diagnostic and therapeutic applications. Additional embodiments of the invention include use of the functionalized elastomeric compositions to detect and isolate target analytes from heterologous fluid mixtures.

### STATEMENT OF RELATED CASES

This International PCT Application claims the benefit of and priority to U.S. Provisional Application No. 63/450,184, filed Mar. 6, 2023, which is incorporated herein by reference in its entirety.

### STATEMENT OF GOVERNMENT INTEREST

This invention was made with government support under grant number 1R21AI154266 awarded by the National Institutes of Health. The government retains certain rights in this invention.

### TECHNICAL FIELD

The invention relates to methods for the use and production of elastomeric particles, such as silicone particles, having functionalized surface domains configured to specifically capture one or more biomarkers.

### BACKGROUND

Particles respond to an applied acoustic standing wave by transporting to specific locations along the wave (i.e., pressure node, pressure antinode). This relocation is dictated by the acoustic contrast factor (i.e., positive contrast, negative contrast), which originates from differences in density and compressibility between the particle and the surrounding media. For example, particles with positive contrast (e.g., incompressible polystyrene beads, cells) in aqueous media are generally transported to acoustic pressure nodes. On the other hand, compressible, elastomeric silicone particles, also referred to herein as negative acoustic contrast particles (NACPs), have a negative acoustic contrast property that is opposite to commonly used particles (e.g., polystyrene beads). Consequently, as shown in FIG. 8, NACPs move to acoustic pressure antinodes when subjected to acoustic standing waves, which is a direction opposite from common, incompressible particles, such as cells in a complex biological sample. When acoustic radiation forces immobilize NACPs along the pressure antinodes, they are herein referred to as “acoustic trapped.”

One drawback of using silicone-based NACPs to relocate target objects (e.g., proteins, cells, nucleic acids) to the pressure antinodes is that they lack a functionalized surface that can be functionalized to specifically capture an object of interest. For example, Lopez et al., discloses in PCT/US2010/034415 the production of stable elastomeric NACPs and their use in acoustic radiation fields, but does not teach the preparation of stable, elastomeric particles having functionalized surfaces that allow for specific binding with biological or chemical ligands. Indeed, Lopez et al. only describe the use of inert silicone (i.e., polydimethylsiloxane (PDMS)) as the starting material to synthesize elastomeric particles without surface functionalization. Effective functionalization of NACPs becomes more important in applications that require high concentrations of active, surface-presenting bio-affinity groups for capturing rare cells, cells with a low quantity of targeted surface antigens, or rare biomolecules from complex biological fluids.

In addition to the use of NACPs for bio-separations in acoustofluidic devices, negative and positive acoustic contrast particles have utility in many industrial fields such as those fields involving the production of paints, foods, inks, coatings, films, cosmetics, and rheological fluids. Using bulk synthetic approaches to synthesize particles with useful biochemical and mechanical properties represents a longstanding goal in synthetic chemistry, chemical engineering, bioengineering, and mechanical engineering. Rapid and scalable synthesis of vast quantities of particles appeals to many industrial fields involving the production of paints, foods, inks, coatings, films, cosmetics, and theological fluids. Such particles also garner importance in scientific communities with examples in the production of slurries, clays, minerals, aerosols, foams, macromolecules, sols, semiconductor nanocrystallites, silica colloids, and biochemical interfaces with proteins, viruses, bacteria, and cells.

As described above, utilization of acoustic contrast particles (also referred to as colloids) in biological and other applications, such as diagnostic screenings or immunological biomarker assays, would require the presence of an effectively functionalized surface adapted to bind a variety of ligands. The ability to rapidly synthesize functionalized NACPs would allow for the identification and acoustic separation of various target ligands, such as biomolecules or chemicals from complex biological solutions.

Accordingly, there remains an unmet need for acoustic contrast particles with functionalized surfaces that would allow for a range of binding and bioconjugation reactions, specifically in complex biological or other samples.

### SUMMARY OF THE INVENTION

In one aspect, the invention includes systems and methods to chemically modify, and specifically functionalize the surfaces of elastomeric particles that can be used in various diagnostic and therapeutic applications.

In another preferred aspect, the invention is directed to systems and methods for producing elastomeric NACPs having a functionalized surface containing a biospecific identification matrix, generally referred to as functional NACPs (fNACPs). In one preferred aspect, the fNACPs of the invention include polydimethylsiloxane (PDMS) particles having a modified surface that enables bio-specific capture of one or more chemical or biological analytes, also interchangeably referred to herein as a target composition.

In another preferred aspect, the invention includes the production of NACPs, and functionalizing the surface of the particles with an identification matrix having one or more stable recognition elements (also referred to as biorecognition elements) that can interact with and bind to a target molecules, preferably in a heterogeneous biological or in vitro solution. In other aspects of the invention, the fNACPs can be isolated using acoustic standing waves to separate the particles from other objects, cells, or biomaterials in a heterogeneous biological sample for the purposes of isolation and detection.

In another preferred aspect, the invention includes bioanalysis of fNACPs. In a preferred aspect, fNACPs can be mixed with a fluid sample, such as a biological sample, suspected of containing target of interest. The fNACPs include a recognition element having a moiety for binding to the target composition, under conditions sufficient that the moiety binds to the target. As shown in FIGS. 6-7, the fluid sample can be subjected to radiation forces from an acoustic standing wave sufficient within an acoustofluidic trapping channel/chamber, which can be part of an acoustic pipette or other similar devices, to focus the particles to the acoustic pressure antinodes such that the target molecules are separated from other components in the sample. The separated fNACPs of the invention can be later removed from the acoustofluidic trapping channel/chamber for further analysis or detection.

In another preferred aspect, the acoustically separated fNACPs can be configured to be “barcoded” to allow for simultaneous and rapid inspection of the fNACPs by standard analytical techniques (e.g., via microscope, plate reader, flow cytometer). Barcoded fNACPs can be coupled with one or varying ratios of multiple fluorescence compositions, such as fluorophores, and/or fluorescent antibodies, and their fluorescence intensity quantified.

In still further embodiments, the invention comprises an assay kit containing quantity of fNACPs, a container and instructions for use.

Additional aspects of the invention may become evident based on the specification and claims presented below.

### BRIEF DESCRIPTION OF THE FIGURES

FIG. 1. Schematic overview of the particle-based, acoustic pipette-enabled assay. negative acoustic contrast particles (fNACPs) capture target biomarkers from biological sample, such as a whole blood sample. The fNACPs are then purified from the blood components by acoustic trapping and captured biomarkers are labeled with a fluorescent antibody within the acoustic pipette. fNACPs fluorescence is then measured to determine biomarker presence and concentration.

FIGS. 2A-C. NACP production. (a) Schematic overview of NACP production. A PDMS precursor is homogenized to form NACP precursor droplets. The solution is heated to facilitate PDMS crosslinking. The cured particles are then filtered while vortexing to isolate a low-polydispersity fraction of NACPs. (b) Brightfield microscopy image of isolated NACPs. Scale bar=100 μm. (c) Histogram of isolated NACP sizes as determined by optical microscopy image analysis.

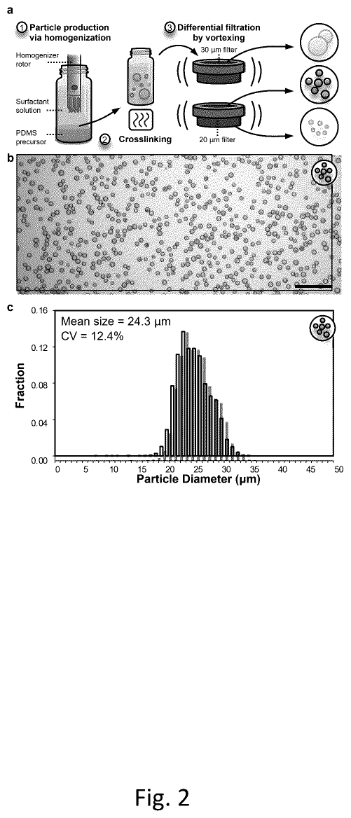

FIGS. 3A-F. NACP trapping and purification. (a) Schematic illustration of acoustic trapping channel including the piezoelectric transducer, capillary channel, and electrical leads. (b) Captured frames from video of trapping of NACPs within an acoustofluidic trapping channel. NACPs, shown in green, were coated with fluorescent streptavidin. Videos were captured by fluorescence microscopy. Scale bar=200 μm. (c) Comparison of NACP retention and sample purity after separation from lysed and unlysed blood samples. Data is presented as mean±standard deviation, N=5. (d) NACP retention over a range of flow rates through the acoustofluidic trapping channel. Data is presented as mean±standard deviation, N=5. (e) Sample purity over a range of flow rates through the acoustofluidic trapping channel. Data is presented as mean±standard deviation, N=5. (f) Comparison of NACP retention and sample purity after separation by automated syringe pump (N=5) or manual operation (N=25). Data is presented as mean±standard deviation. Data for manual operation is presented as the mean of results from 5 individual operators, each with 5 individual samples.

FIGS. 4A-D. fNACP functionalization. (a) Schematic illustration of fNACP functionalization approach. b Overlaid histograms of fluorescent streptavidin (FSA) fluorescence intensity of fNACPs without FSA, incubated with blocked FSA, incubated with FSA and without BPT (biotin-PEG-triethoxyvinylsilane), and functionalized as normal. Data is presented on a biexponential x-axis. (c) Fluorescence microscopy image of fNACPs functionalized with FSA. Scale bar=100 μm. (d) Comparison of NACP FSA fluorescence intensity with and without acoustic separation. Data is presented as median±standard deviation, N=5.

FIGS. 5A-D. fNACP assay overview and analysis. (a) Schematic illustration of a standalone NACP-based assay for anti-ovalbumin (OVA) detection. Capture fNACPs and control NACPs are incubated, separated, and analyzed simultaneously. (b) fNACP fluorescence intensity of blank fNACPs, fNACPs incubated with only fluorescent anti-IgG, and fNACPs incubated with fluorescent anti-IgG after incubation with 0.1 nM anti-OVA. Data is presented as average mean fluorescence intensity (MFI)±standard deviation. N=3, *P<0.05. (c) Capture fNACP and control NACP anti-IgG fluorescence over range of anti-OVA concentrations spiked in buffer. Data was fit to a 5-parameter logistic curve fit (5PL) curve to determine the assay limit of detection (LOD) and lower limit of quantitation (LLOQ). Data is presented as mean MFI±standard deviation. N=3. (d) Overlaid fluorescence microscopy images of control NACPs and capture fNACPs after incubation with fluorescent anti-IgG after incubation with 5×10⁴ nM anti-OVA. Images were captured using green and red fluorescence channels. Scale bar=50 μm.

FIGS. 6A-B. Acoustic pipette overview. (a) Image of an acoustic pipette to trap NACPs. Inset shows mechanism for movement and locking of trapping chamber. (b) Schematic illustration of acoustic pipette internals, including the main pipette body and trapping chamber. c Schematic illustration of general pipette-facilitated fNACP assay.

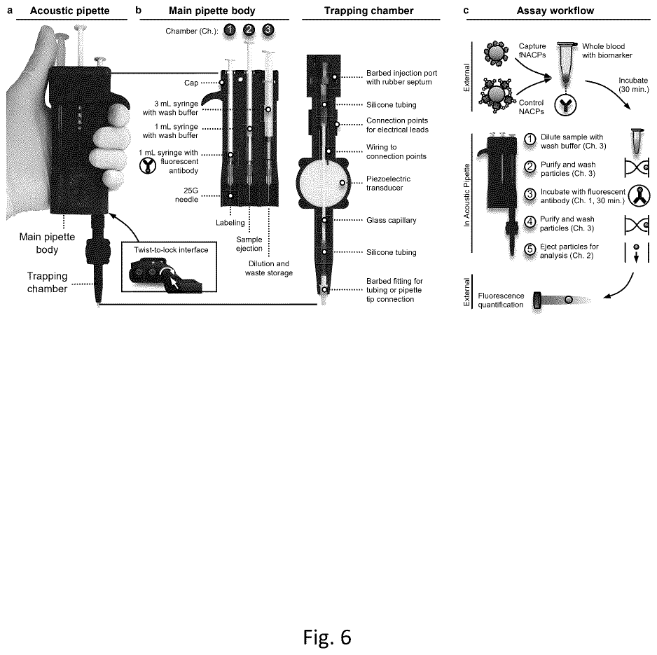

FIGS. 7A-B. Detection of anti-OVA from whole blood in the acoustic pipette. (a) Schematic illustration of detection of anti-OVA from whole blood via the acoustic pipette. (b) Capture fNACP and control NACP anti-IgG fluorescence over range of anti-OVA concentrations spiked in whole blood. Data was fit to a 5PL curve to determine the assay LOD and LLOQ. Data is presented as mean MFI±standard deviation. N=3.

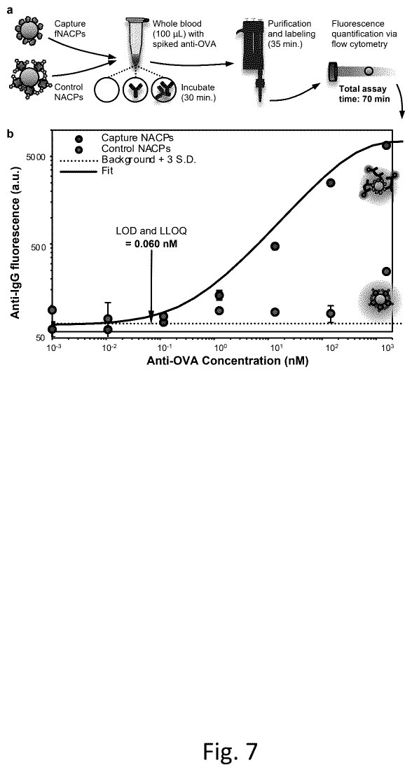

FIG. 8 shows a schematic representation of the acoustic separation of fNACPs (i.e., biomarker-specific NACPs) from a heterogeneous biological sample in an acoustofluidic trapping channel.

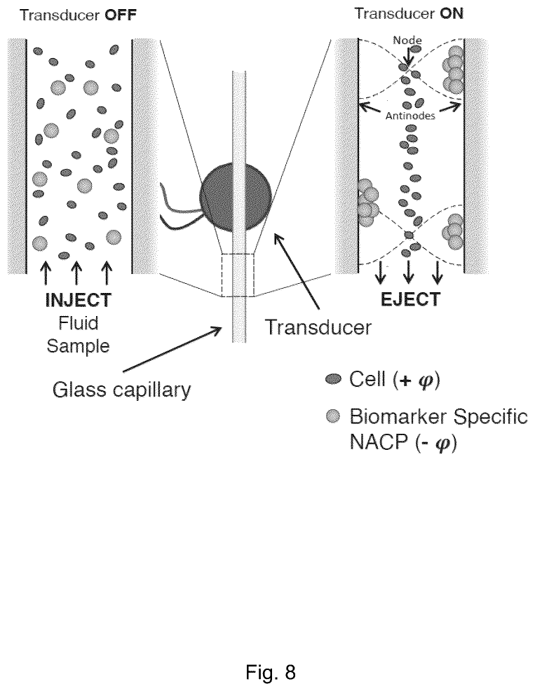

### DETAILED DESCRIPTION OF THE INVENTION

The present invention is directed to systems, methods, and compositions for capturing and identifying one or more target compositions (also referred to herein as analytes or molecules), which can include biological or chemical ligands. In a preferred aspect, the invention is directed to the novel use of negative acoustic contrast particles (NACPs) that have been functionalized to capture one or more target compositions, preferably in heterogeneous biological samples that can further be acoustically separated. In one embodiment, the invention includes compositions for, and method of producing functionalized NACPs (fNACPs) having a recognition element configured to interact and bind with its cognate target composition, which can include biological or chemical ligands, and preferably a biomarker. For example, in one aspect, a target composition of the invention can include, but is not limited to: cell, a protein, a receptor, an antibody, an antigen, an aptamer, a drug, a virus, a bacterium, a nucleic acid, a polysaccharide, a drug, an analyte, a metabolite, or a moiety or a combination of the same.

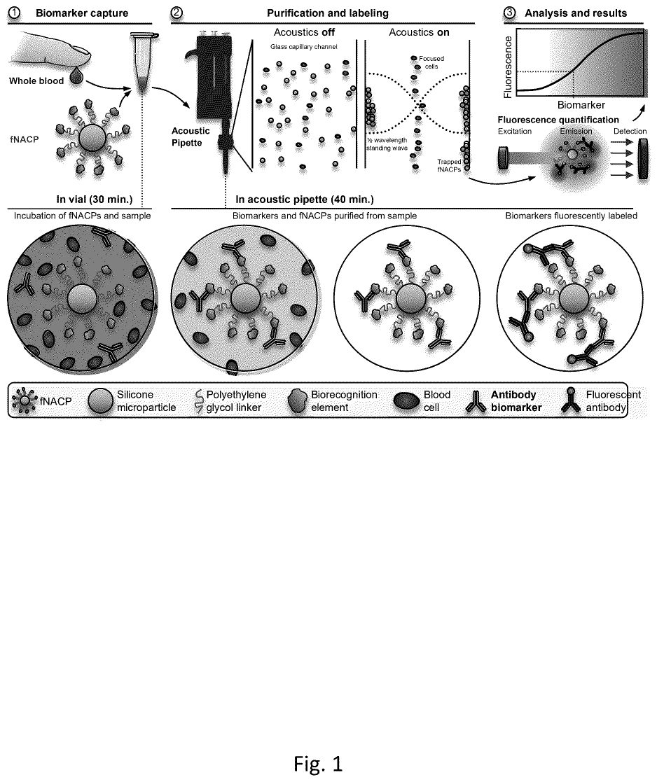

Generally referring to FIG. 1, the fNACPs of the invention can be configured to include bio-specificity for the identification and capture of a target composition, such as a biomarker, from a complex biological sample. In this embodiment, fNACPs of the invention can be added to a fluid, such as a complex biological sample and bind to a target composition, such as a biomarker within the sample. In this embodiment, the biological sample and fNACPs of the invention can be acoustically separated, thereby isolating the bound target compositions from the sample.

Again, referring to FIG. 1, in a preferred embodiment, the acoustically separated fNACPs can be configured to be “barcoded” to allow simultaneous and rapid inspection of the fNACPs by standard analytical techniques (e.g., via microscope, plate reader, flow cytometer). Barcoded fNACPs having unique identifiers further allows multiple target compositions to be probed in the same experimental procedure. In a preferred embodiment, methods and systems of barcoding fNACPs can include measuring fluorescence intensity with varying ratios of multiple fluorophores, such as fluorescent antibodies or proteins conjugated to the fNACPs.

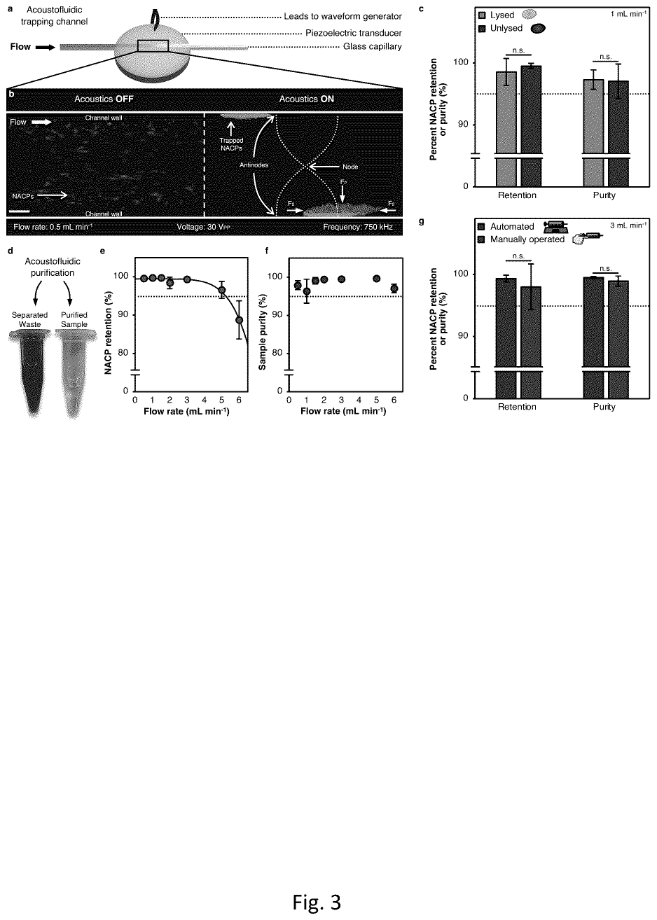

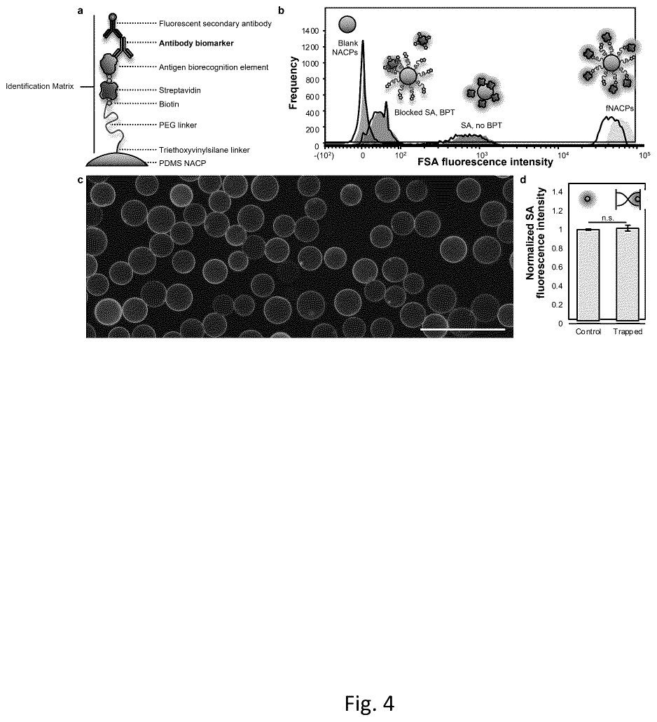

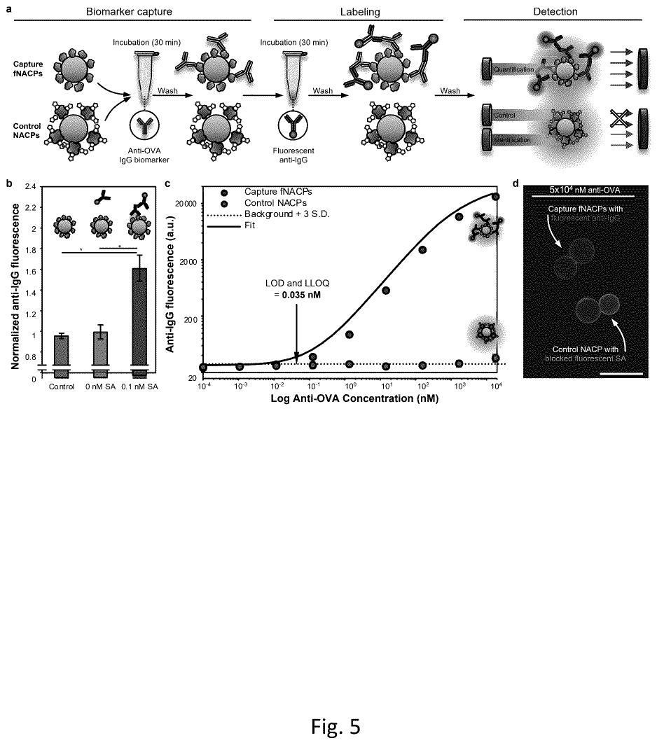

As shown in FIGS. 3-4, target compositions bound to fNACPs can be detected and quantified through the excitation of a fluorescent antibody that is further bound to the target composition of interest. In this manner, the fNACPs of the invention can be fluorescently “barcoded” to capture and report a range of target compositions, such as biomarkers in a biological sample. In certain embodiments, the binding of a small number of differently colored fluorophores to various fNACPs, or more preferably to the target compositions bound to the fNACPs, can provide discriminative potential through variations in relative concentrations of each molecule in a sample. For example, attachment three different fluorophores at 8 different concentrations yields over 500 distinct barcodes which can be uniquely detected and attributed to a specific fNACP. Fluorometric barcoding of fNACP particles is easily achieved through attachment of fluorophores conjugated to antibodies, reactive groups, or any other moiety which can be bound to the surface of the fNACP, as generally shown in FIGS. 4-5. General methods and systems of “barcoding” compositions, such as NACPs for bioassays have been generally described by Yang M, et al., in barcoded point-of-care bioassays. Chem Soc Rev. 2019; 48(3):850-84. The specific methods, devices, and systems described therein, as applicable to the fNACPs of the present invention, as would be understood by one or ordinary skill in the art, are incorporated herein in their entirety by reference.

In a preferred embodiment, the invention includes a NACP, preferably formed of elastomeric silicone, such as polydimethylsiloxane (PDMS). Referring now to FIG. 4A, the surface of the NACP can be functionalized to include an identification matrix having a recognition element that can interact and bind to one or more analytes in a fluid solution, and preferably a heterogeneous solution such as a complex biological solution containing a number of different analytes, ligands, or other biological materials. Again, referring to FIG. 4A, the identification matrix of the invention includes a triethoxyvinylsilane (TEOVS) linker coupled to the surface of the NACP. The TEOVS linker can further be coupled with a linker (e.g., an antifouling compound, such as polyethylene glycol (PEG)). In a preferred embodiment, the PEG linker can be tethered to the TEOVS linker on one end, and a specific binding domain, such as biotin compound on the other end.

The size of the PEG linker can be variable. Specifically, different sized PEG molecules can exhibit distinct antifouling properties. Moreover, having a PEG linker of sufficient length can allow the biorecognition element to be sterically unhindered and thereby allow it to form more favorable orientations for enhanced target molecule capture. In certain embodiment, the length of the PEG linker domains of the identification matrix can be uniform, while in alternative embodiments, PEG linker domains of the identification matrix can have different sizes forming a heterogeneous functionalized surface. In this configuration, the biorecognition element can have different binding characteristics compared to a homogenous functionalized surface.

Referring again to FIG. 4A, the biotin domain can further be complexed with a biotin-binding protein, such as streptavidin or avidin. As further shown in FIG. 4A, a recognition element or domain can also be bound to a biotin compound, allowing it to also complex with the streptavidin protein, thereby anchoring the recognition element to the surface of the NACP through the streptavidin-biotin-PEG-TEOVS construct.

In a preferred embodiment, the biorecognition domain of the invention comprises a chemical or biological domain having one or more moieties configured to bind to a target analyte, such as a biomarker. In the preferred embodiment shown in FIG. 4, the biorecognition domain of the invention includes an antigen having a moiety that allows it to bind to a corresponding antibody. However, additional embodiments can include a variety of biorecognition domains having one or a plurality of moieties that bind to one or more target analytes. For example, in certain embodiments, the biorecognition domain can include, but not be limited to a protein, a receptor, an antibody, an antigen, an aptamer, a nucleic acid, a polysaccharide, a drug, an analyte, a metabolite, or a functional fragment, preferably continuing a binding moiety or a combination of the same.

Moreover, in certain embodiments, a fNACP of the invention may be homogenously functionalized with an identification matrix having a uniform biorecognition domain configured to detect and bind to a single target analyte. In alternative embodiments, a fNACP of the invention may be heterogeneously functionalized with a plurality of identification matrixes having a different biorecognition domains configured to detect and bind to a plurality of target analytes in a fluid sample.

As shown in FIGS. 1 and 4, a quantity of fNACPs having biorecognition domains for one or more target molecules can be contacted with a fluid sample. The fluid sample containing the fNACPs can be subjected to acoustic radiation forces from an acoustic standing wave within an acoustofluidic trapping channel/chamber to focus the particles to the pressure antinodes such that the target molecules, bound to the surfaces of the fNACPs, are separated from other components in the sample without disrupting the bonds of the recognition element-streptavidin-biotin-PEG-TEOVS construct on the surfaces of the particles, thereby preventing the loss or disruption of the identification matrices and bound target analyte(s).

The fNACP of the invention, after capturing a target molecule, such as a biomarker in a sample, can further be labeled, preferably with a florescent label to allow rapid identification of the same. As further shown in FIG. 5, a fNACP bound to a target analyte can be labeled with, in this case a fluorescent antibody configured to bind to one or more moieties of the target. The labeled fNACP can be detected by excitation of the florescent label and compared to, for example, unlabeled control NACPs that lack the recognition binding domain, and which may contain, or be bound to a differentially colored fluorophore and the like.

The fNACPs of the invention can be removed from the acoustofluidic trapping channel/chamber for further analysis or detection. As noted above, in certain embodiments, the fNACPs of the invention can be “barcoded” to allow to allow simultaneous and rapid inspection of the fNACPs. Barcoded fNACPs can be coupled with one or varying ratios of multiple fluorescence compositions, such as fluorophores, and/or fluorescent antibodies, and their fluorescence intensity quantified. In one embodiment, a mixture of fluorescent streptavidin can be used during the functionalization thereby barcoding the NACPs.

The invention further provides methods of producing and functionalizing the surface of an NACP. As described in the Examples below, bonding of the triethoxyvinylsilane (TEOVS) linker to the surface of the NACP yields surface-exposed silane or silanol groups which can form covalent siloxane bonds with the silane groups of the biotin-PEG-silane (BPS). In this manner, Applicant's couple BPS to the particles first to yield particles with covalently bound (BPS). In this example, streptavidin (SA) can bind to the biotin on the end of the BPS, and since the SA has 4 biotin binding sites, one or more biotinylated recognition elements can be coupled with the SA and presented for target molecule capture. As such, each functionalization step builds on the last and provides a new functional group/binding motif for the following step. As detailed description of the same is provided below:

### NACP Production

The present invention describes the production of NACPs. In one embodiment, a base of Sylgard 184 base and cross-linker was mixed with a quantity of triethoxyvinylsilane (TEOVS). A surfactant, such as Pluronic/F-108 was added to the mixture and homogenized and incubated overnight. After the particles have formed and cooled, they are redispersed. In one preferred embodiment, the present invention describes the production of NACPs according to the following exemplary procedure:
1. Add 2.0 g of Sylgard 184 base to 20 mL scintillation vial.
2. Add 0.2 g of Sylgard 184 cross-linker to the vial.
3. Add 0.1% v/w (2.2 uL) triethoxyvinylsilane (TEOVS).
4. Mix thoroughly with a metal stir bar.
5. Add 18 mL of 1% Pluronic/F-108 solution to the vial.
6. Homogenize with at a setting of 4 out of 6 for 1 minute. Make sure that all PDMS at the bottom of vial is homogenized. If PDMS is stuck at bottom, lightly swirl the vial with the homogenizer tip in contact with corners of vial.
7. Add a stir bar and stir at 300-400 rpm for 2 hours at 75° C. (alternatively, stir overnight at 65° C.).
8. Redisperse the cooled particles.

### NACP Separation

The present invention describes the production of NACPs. In one embodiment, the particles generated from the process described in Example 1, were pass through a series of filters while being vortexed continuously, and further washed with a surfactant, such a Pluronic solution. In one preferred embodiment, the present invention describes the separation of NACPs according to the following exemplary procedure:
9. Place a 40-micron cell strainer over a 50 mL centrifugation tube.
10. Pour approximately 5 mL of the particles through the strainer while vortexing continuously.
11. Flush the strainer with ~3 mL 1% Pluronic solution while continuing to vortex.
12. Invert the strainer, flush into waste beaker with deionized water (DIW) or Pluronic solution.
13. Repeat steps 9-12 until all particles have been filtered.
14. Repeat steps 9-13 with a 30-micron cell strainer and connector until all particles have been filtered.
15. Repeat steps 9-13 with a 20-micron filter and connector.
16. After filtering a fraction of the particles, invert filter and flush the solution into a 50 mL tube with ~3 ml of 1% Pluronic solution while vortexing to collect particles between the 20- and 30-micron filters.
17. Repeat 15-16 until all particles have been collected.
18. Optionally image and size particles.

### fNACP Functionalization

The present invention describes the functionalization of NACPs to form fNACPs. In one embodiment, the particles separated by the process described in Example 2, were washed and incubated with a quantity of biotin-PEG-silane (BPS). Next, a quantity of silane-PEG-biotin was dissolved in the particle/BPS solution and mixed. A buffer and additional surfactant, in this embodiment a solution of phosphate-buffered saline (PBS) and Tween 20, was added to the mixture which underwent multiple rounds of centrifugation and aspiration. The centrifuged pellet was resuspended in a streptavidin (SA) solution. Separately, a quantity of desiccated SA was added to a PBS solution and aliquoted into separate containers and frozen. The particle solution went through additional resuspension by vertexing followed by washing with a PBS and Tween 20 solution followed again by centrifugation. The isolated particles were then counted, in this case using a hemocytometer, and resuspended in a buffered solution containing an exemplary biotin-OVA, which was again repeatedly washed, centrifuged, and resuspended. The now functionalized NACPs (i.e., fNACPs) were dispersed in a solution of ELISA-grade BSA in PBS, counted again, and resuspended in a wash buffer and stored for later use.

In one preferred embodiment, the present invention describes the functionalization of NACPs according to the following exemplary procedure:
19. Remove desired samples and distribute in centrifuge tubes.
20. Wash all samples twice (3000×g, 3 min), resuspending in DIW with 0.004% Tween 20.
21. Incubate with BPS solution in an Eppendorf tube. a. For a sample of 5 million particles, dissolve 1.5 mg silane-PEG-biotin in 50 μL DIW.
   b. Add to samples.
   c. Mix for 2 hours on VorTemp mixer at 1000 rpm. Ensure that the particles remain suspended. Occasionally increase speed to resuspend if necessary.
22. Add 900 uL PBS with 0.004% Tween 20.
23. Centrifuge at 3000×g for 3 minutes.
24. Aspirate.
25. Repeat steps 22-24 once.
26. Repeat step 22 once.
27. Transfer to a new Eppendorf tube.
28. Repeat steps 23 and 24 once.
29. Repeat steps 22-24 once (total: 4 centrifuge steps).
30. Resuspend/add to pellet with 10-100 μL streptavidin (SA) solution per 5 million particles. a. Add 30 μL PBS per 10 μL SA used. Pipette up and down to disperse.
   b. Dissolve 1 mg desiccated SA to 0.6 mL PBS.
   c. Aliquot 100 μL volumes SA into sterile Eppendorf vials and store at −20° C.
31. Mix for 90 minutes on VorTemp mixer at 1150 rpm. Ensure that the particles remain suspended.
32. Add 900 uL PBS with 0.004% Tween 20.
33. Centrifuge at 3000×g for 3 minutes.
34. Pour the decant into a waste container.
35. Repeat steps 32-34 once.
36. Add 1000 uL PBS with 0.004% Tween 20.
37. Transfer to a new Eppendorf tube.
38. Count the particles using hemocytometer.
39. Repeat steps 33 and 34 once (total: 4 centrifuge steps).
40. Resuspend/add to pellet with 0.01 mg-0.1 mg biotin-OVA per 1 million particles. (Solution is 1 mg/mL in PBS per sample).
41. Mix for 2 hours on VorTemp mixer at 1000 rpm. Ensure that particles remain suspended. Occasionally increase the speed to resuspend if necessary.
42. Add 900 uL PBS with 0.004% Tween 20.
43. Centrifuge at 3000×g for 3 minutes.
44. Pour the decant into a waste container.
45. Repeat steps 42-44 once (total: 2 centrifuge steps).
46. Disperse thoroughly in 1000 μL wash buffer (1% (10 mg/mL) ELISA-grade BSA in PBS).
47. Count the particles using hemocytometer.
48. Repeat steps 43 and 44 once.
49. Resuspend in 1.5 mL wash buffer and store in the fridge.

As should be noted, the above examples outline process steps that are exemplary in nature and should in no way be considered limiting. For example, in certain embodiment one or more steps may be omitted or the order of such steps changed or modified, without departing from the methods claimed in the invention.

Unless defined otherwise, all scientific and technical terms are to be understood as having the same meaning as commonly used in the art to which they pertain.

The target compositions, molecules or markers of this invention may be used for diagnostic and prognostic purposes, as well as for therapeutic, drug screening, and patient stratification purposes (e.g., to group patients into a number of “subsets” for evaluation), as well as other purposes described herein.

As used herein, a biological marker (i.e., a “biomarker”, a “marker”, or an “analyte”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process.

The present invention also encompasses recognition domains or elements, the two being generally interchangeable, which specifically bind the target molecules. As used herein, the term “binding” refers to the interaction between binding pairs (e.g., an antibody and an antigen or aptamer and its target). In other embodiments, the phrase “binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contains antibody or antigen binding fragments alone (i.e., in the absence of antigen), wherein an amount of reactivity (e.g., non-specific binding to the well) by the antibody or antigen binding fragments thereof in the absence of the antigen is considered to be background. Binding can be measured using a variety of methods standard in the art including enzyme immunoassays (e.g., ELISA, immunoblot assays, etc.).

The molecules that may bind to one or more of the targets include antibodies, aptamers and antibody derivatives or fragments. As used herein, the term “antibody” refers to an immunoglobulin molecule capable of binding an epitope present on an antigen. The term is intended to encompass not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity.

As used herein, an aptamer is a non-naturally occurring nucleic acid molecule or peptide having a desirable action on a target, including, but not limited to, binding of the target, catalytically changing the target, reacting with the target in a way which modifies/alters the target or the functional activity of the target, covalently attaching to the target as in a suicide inhibitor, facilitating the reaction between the target and another molecule. In one embodiment, the antibodies, antibody derivatives or fragments, or aptamers specifically bind to a component that is a fragment, modification, precursor, or successor of one or more target molecules.

As used herein, “sample” includes a quantity of fluid containing a target molecule. As further used herein, the term “biological sample” includes a sample from any bodily fluid or tissue (e.g., serum, plasma, blood, cerebrospinal fluid, urine, saliva, cancer tissue, healthy tissue), preferably from an animal, and more preferably from a mammal, and even more preferably from a human subject.

As used herein, the term “nucleic acid” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, “silane” refers to a silicon-containing group having the formula SiR₄, where each R group can be alkyl, alkenyl, cycloalkyl, phenyl, or other silicon-containing groups. When the silane is linked to another compound, the silane is referred to as a “silyl” and has the formula—SiR₃. Further, a “siloxane bond” means a molecular structure in which silicon (Si) and oxygen (O) are bonded.

As used herein, “biotinylated” means that a substance is conjugated to one or more biotin moieties. Biotinylated peptides useful in practicing the invention can be purchased commercially (e.g., Midwest Bio-Tech Inc.) or can be readily synthesized and biotinylated. Biotinylation of compounds, such as peptides, can be by any known chemical technique. These include primary amine biotinylation, sulfhydryl biotinylation, and carboxyl biotinylation. For example, amine groups on the peptide, which are present as lysine side chain epsilon-amines and N-terminal α-amines, are common targets for primary amine biotinylation.

As used herein, a composition is referred to as “isolated” when it has been separated from at least one component with which it is naturally associated. For example, a fNACP can be considered isolated if it is separated from cellular components present in a sample, such as a biological sample, including cells, organelles, polysaccharides, lipids, polypeptides, polynucleotides, and other metabolites. Standard quantification methodologies known in the art can be employed to obtain and isolate the molecules of the invention.

As used herein the singular forms “a”, “an”, and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1 % compared to the specifically recited value.

1. A functionalized negative acoustic contrast particle (fNACP) comprising:
a negative acoustic contrast particle (NACP) particle having a triethoxyvinylsilane (TEOVS) linker bound to the surface of the particle generating a surface-exposed silane or silanol group;
an antifouling agent forming a siloxane bond with the surface-exposed group; and
a recognition element operably linked to said antifouling agent.

2. The particle of claim 1, wherein the particle comprises an elastomeric particle.

3. The particle of claim 2, wherein said elastomeric particle selected from: a silicone particle, or a polydimethylsiloxane (PDMS) particle.

4. The particle of claim 1, wherein said antifouling agent comprises a polyethylene glycol (PEG) molecule coupled with a first biotin.

5. The particle of claim 4, wherein said first biotin is coupled with a streptavidin (SA) or avidin molecule.

6. The particle of claim 5, wherein said recognition element comprises a biotinylated recognition element bound to said SA or said avidin molecule, and configured to bind a target analyte.

7. The particle of claim 6, wherein said biotinylated recognition element is selected from: a biotinylated protein, a biotinylated receptor, a biotinylated antibody, a biotinylated antigen, a biotinylated aptamer, a biotinylated nucleic acid, a biotinylated polysaccharide, a biotinylated drug, a biotinylated analyte, a biotinylated metabolite, or a biotinylated fragment or moiety, or a combination of the same.

8. (canceled)

9. The particle of claim 6, wherein said target analyte is selected from: a cell, a protein, a receptor, an antibody, an antigen, an aptamer, a drug, a virus, a bacterium, a nucleic acid, a polysaccharide, a drug, an analyte, a metabolite, a small molecule, or a fragment or moiety, or a combination of the same.

10. The particle of claim 1, and further comprising wherein said fNACP is barcoded.

11. The particle of claim 6, and further comprising an antibody coupled with a moiety of said target analyte.

12. The particle of claim 11, wherein said antibody comprises a fluorescent antibody, or antibody conjugated with one or more tags configured to enable antibody detection and/or quantification, or antibody conjugated with one or more tags configured to enable antibody detection or quantification in the presence of a substrate.

13-14. (canceled)

15. A functionalized negative acoustic contrast particle (fNACP) comprising:
an elastomeric particle having an identification matrix bound to its surface, the matrix comprising:
a triethoxyvinylsilane (TEOVS) linker bound to the surface of the particle generating a surface-exposed silane or silanol group;
a linker comprising a polyethylene glycol (PEG) molecule coupled with a first biotin molecule, wherein said PEG molecule forms a covalent siloxane bond with the surface-exposed group;
a streptavidin (SA) molecule bound to said first biotin; and
one or more biotinylated recognition elements bound to said SA, and configured to bind a target analyte.

16-26. (canceled)

27. A system comprising:
a fluid sample containing a target analyte;
a functionalized negative acoustic contrast particle (fNACP) having an identification matrix bound to its surface, the matrix comprising:
a triethoxyvinylsilane (TEOVS) linker bound to the surface of the particle generating a surface-exposed silane or silanol group;
a linker comprising a polyethylene glycol (PEG) molecule coupled with a first biotin molecule, wherein said PEG molecule forms a covalent siloxane bond with the surface-exposed group;
a streptavidin (SA) molecule bound to said first biotin;
one or more biotinylated recognition elements bound to said SA, and configured to bind the target analyte; and
an acoustic separator.

28. The system of claim 27, wherein said fNACP comprise a functionalized polydimethylsiloxane (PDMS) particle.

29. The system of claim 27, wherein said biotinylated recognition element comprises a biotinylated protein, a biotinylated receptor, a biotinylated antibody, a biotinylated antigen, a biotinylated aptamer, a biotinylated nucleic acid, a biotinylated polysaccharide, a biotinylated drug, a biotinylated analyte, a biotinylated metabolite, or a biotinylated fragment or moiety or a combination of the same.

30-32. (canceled)

33. The system of claim 27, wherein said target analyte is selected from: a cell, a protein, a receptor, an antibody, an antigen, an aptamer, a drug, a virus, a bacterium, a nucleic acid, a polysaccharide, a drug, an analyte, a metabolite, a small molecule, or a fragment or moiety, or a combination of the same.

34. The system of claim 27, wherein said fNACP is barcoded, or wherein said fNACP comprises two or more fNACPs having a unique recognition element that are further individually barcoded so as to correspond to said unique recognition element.

35. The system of claim 27, and further comprising an antibody coupled with a moiety of said target analyte.

36. The system of claim 35, wherein said antibody comprises a fluorescent antibody, or antibody conjugated with one or more tags configured to enable antibody detection, or quantification, or an antibody conjugated with one or more tags configured to enable antibody detection or quantification in the presence of a substrate.

37. The system of claim 27, wherein said acoustic separator comprises a device configured to generate an acoustic standing wave to focus the particles to the acoustic pressure antinodes.

38-66. (canceled)
